(19) United States
(12) Patent Application Publication
(10) Pub. No.: US 2023/0067474 A1
(43) Pub. Date: Mar. 2, 2023

(54) SLEEP APNEA MOUTH GUARD SENSOR

(71) Applicants: Yixin Wang, Basking Ridge, NJ (US); David Xudong Wang, San Diego, CA (US)

(72) Inventors: Yixin Wang, Basking Ridge, NJ (US); David Xudong Wang, San Diego, CA (US)

(21) Appl. No.: 17/887,066

(22) Filed: Aug. 12, 2022

Related U.S. Application Data
(60) Provisional application No. 63/236,753, filed on Aug. 25, 2021.

(51) Int. Cl. A61B 5/11; A61B 5/0205; A61B 5/00; A61B 5/08

(52) U.S. Cl. CPC A61B 5/11; A61B 5/0051; A61B 5/0205; A61B 5/0826; A61B 5/4818

(57) ABSTRACT

The present invention is directed to a sensor device detecting movement of mouth or tongue associated with bruxism and/or sleep apnea. The present invention is also directed to a system for treating sleep apnea in a patient in need thereof, comprising a sensing component and a stimulation component, the sensing component comprising one or more wireless sensors for collecting one or more vital signs of the patient and/or tongue muscle movements of the patient, the sensing component being in wireless communication with a control system, and wherein the stimulation component comprises (i) an implantable body configured to deliver energy to one of a nerve or muscle, and (ii) a wearable appliance inductively coupled to the implanted body, the wearable portion configured to receive signals from the control system, wherein the sensing component comprises the sensor device of the present invention.

### CROSS-REFERENCE TO RELATED APPLICATIONS

This application claims the priority benefit of U.S. Provisional Application No. 63/236,753, filed Aug. 25, 2021, which is incorporated herein by reference in its entirety.

### TECHNICAL FIELD

The present invention relates to a device capable of detecting togue movement associated with sleep apnea and/or bruxism. The present invention also relates to a system for treating sleep apnea comprising such a device.

### BACKGROUND OF THE INVENTION

Sleep apnea is a sleep disorder in which pauses in breathing or periods of shallow breathing during sleep occur more often than normal. Each pause can last for a few seconds to a few minutes and they happen many times a night. In the most common form, this follows loud snoring. Symptoms include snoring loudly and feeling tired even after a full night’s sleep. Treatment often includes lifestyle changes, such as weight loss, and the use of a breathing assistance device at night, such as a continuous positive airway pressure (CPAP) machine.

Detecting tongue/mouth movement is essential to monitor and/or treating sleep apnea. It is desirable in the art to find devices that are convenient, safe, cost-effect and sensitive in detecting mouth/tongue movement. The present inventions are directed to a novel electronic vibration sensor capable of detecting togue movement associated with sleep apnea and/or bruxism. This sensor offers significant advantages, including small package size, low cost, no data processing, and high selectivity/sensitivity in detecting sleep apnea associated with togue movement.

### SUMMARY OF THE INVENTION

The present invention is directed to a sensor device detecting movement of mouth or tongue associated with bruxism and/or sleep apnea.

In one aspect, the device is capable of filtering at least 80%, 90%, or 95% mouth or tongue movement not associated with bruxism and/or sleep apnea.

In one aspect, the device comprising an electronic sensor.

In one embodiment, the electronic sensor is a vibration sensor, preferably a high sensitivity sensor.

In one embodiment, the sensor is coupled with a vibration causing switch, which is further connected with a power source.

In an exemplary embodiment, the device is the sensor shown in FIG. 3, and/or device has a prototype design shown in FIG. 4.

In one aspect, the device is wearable.

The present invention is directed to a method of detecting tongue or mouth movement associated with bruxism and/or sleep apnea, comprising 1) putting the device shown in FIG. 4 in to the mouth of a subject, and 2) collecting signals shown in the device.

The present invention is also directed to a system for treating obstructive sleep apnea in a patient in need thereof, The system comprises a sensing component and a stimulation component, the sensing component comprising one or more wireless sensors for collecting one or more vital signs of the patient and/or muscle movement of the patient, the sensing component being in wireless communication with a control system, and wherein the stimulation component comprises (i) a implantable body configured to deliver energy to one of a nerve or muscle, and (ii) a wearable appliance inductively coupled to the implanted body, the wearable portion configured to receive signals from the control system.

In one aspect, the sensing component comprises the sensor device of the present invention.

In one embodiment, the vital signs are selected from the group consisting of blood oxygen, respiration rate, and heart rate. The muscle movement is tongue muscle movement.

In another embodiment, the wearable portion is a dental appliance comprising a rechargeable battery, a pulse Hebert, and a means for inductively delivering electronic ferry to the implantable body.

In one embodiment, the means of inductively delivering energy is a transmitter coil.

In another embodiment, the implantable body comprises a receiver coil for receiving energy from the wearable portion.

In one embodiment, the implantable body is configured to deliver energy to a hypoglossal nerve.

In one embodiment, the wearable portion is a dermal device for positioning on the patient’s skin, and wherein the dermal device comprises a means for inductively delivering energy to the implantable body.

In one embodiment, the implantable body is configured to deliver energy to a geniohyoid muscle.

In another embodiment, the implantable body comprises a means for wirelessly receiving energy from the dermal device, and wherein the implantable device further comprises an insulating disc.

In one aspect, the control system is embedded within the wearable apparatus of the stimulation component.

In one embodiment, the control system having a first member of a pair of inductive power transfer coils, a wearable apparatus having a second member of a pair of inductive power transfer could, a rechargeable battery, and a pulse generator, wherein the wearable apparatus is configured to wirelessly deliver energy to the implantable body upon receipt of a signal inductive of a sleep apnea event; and wherein the implantable body is configured to transfer the energy received from the wearable apparatus to a hypoglossal nerve or a geniohyoid muscle positioned in proximity.

In another embodiment, the wearable apparatus is a dental appliance adapted for placement over the patients’ lower teeth.

In another embodiment, the dental appliance is a bite splint or a retainer.

In an exemplary embodiment, the wearable apparatus further comprises means for receiving control signals from a control system communicatively coupled thereto.

In an exemplary embodiment, the control system of the wearable apparatus comprises a processor, a memory, and a wireless communications module, the control system configured to (i) receive signals from one or more wireless sensors, (ii) process the signals to determine if a sleep apnea event has occurred or will occur, and (iii) send control signals to the wearable apparatus.

In another exemplary embodiment, the apparatus control system is embedded within the dental appliance.

The present invention is directed to a system for treating sleep apnea in a patient in need of treatment thereof comprising (i) one or more wireless sensors, (ii) a stimulation device, a stimulation device having a wearable portion and an implantable portion, the wearable portion configured to wirelessly transmit stimulation pulses to the implant e portion, and (iii) a control system, the control system having a memory coupled to one or more processors, the memory to store computer-executable instructions that, when executed by one or more processors, cause one or more processors to perform operations comprising (a) measuring vital signals of a patient using one or more wireless sensors; (b) determining whether a sleep apnea event has occurred or will occur based on the measured vital signals; (c) facilitating the delivery of a stimulation pulse to treat sleep apnea using the stimulation component; wherein the control system is in wireless communication with both the one or more wireless sensors and the wearable portion of the stimulation component.

In one aspect, the measured vital signs are used to derive a sleep apnea index, and wherein the step of determining whether the sleep apnea event has occurred or will occur comprises comparing the derived sleep apnea index Tia pre-determined sleep apnea index specific for the patient, the pre-determined sleep apnea index being stored in the memory.

In another aspect, one or more wireless sensors include a respiration sensor, and wherein the step of determining whether the sleep apnea event has occurred or will occur comprises comparing measured respiration rates to a pre-determined threshold respiration rate.

In yet another aspect, the wearable apparatus is a dental appliance, and wherein the dental appliance comprises at least two transmission coils for delivering the stimulation pulses to two implantable bodies, the dental appliance adapted to releasable engage a portion of the patient’s lower teeth, the at least two transmission coils positioned on an exterior surface of the dental appliance.

In yest another aspect, the present invention is directed to a method of treating sleep apnea in a patient with the system disclosed herein.

### BRIEF DESCRIPTION OF THE DRAWINGS

FIG. 1 shows one particular vibration sensor switch test set up.

FIG. 2A shows a perspective view of one particular design of the sleep apnea mouth guard sensor device of the invention.

FIG. 2B shows a different perspective view of particular design of the sleep apnea mouth guard sensor device of the invention shown in FIG. 2A.

FIG. 2C shows a perspective view of a different design of the sleep apnea mouth guard sensor device of the invention.

FIG. 3 is a photograph of Exemplary Fast Vibration Sensor Switch.

FIG. 4 is a photograph of Exemplary Medium Vibration Sensor Switch.

FIG. 5 is a photograph of Exemplary Slow Vibration Sensor Switch.

FIG. 6 is a photograph of Digi-Key Part Number: 1670-1082-ND.

FIG. 7 is a photograph of Digi-Key Part Number: P031-ND.

FIG. 8 is a photograph of Digi-Key Part Number: 36-3030-ND.

FIG. 9 summarizes lists performance characteristics.

FIG. 10 shows one particular CPAP device as an example of mechanical based sensing devise.

FIG. 11 is a summary of electronical sensors.

### DETAILED DESCRIPTION OF THE INVENTION

The present inventions are directed to a novel device detecting month or togue movement associated with sleep apnea and/or bruxism. In a preferred embodiment, the movement is only associated with sleep apnea, including tongue flicking, quick head nodding, quick head shaking, teeth grinding, and is not associated with normal sleep movement. Including, but not limited to, swallowing, slow head nodding, slow head shaking,

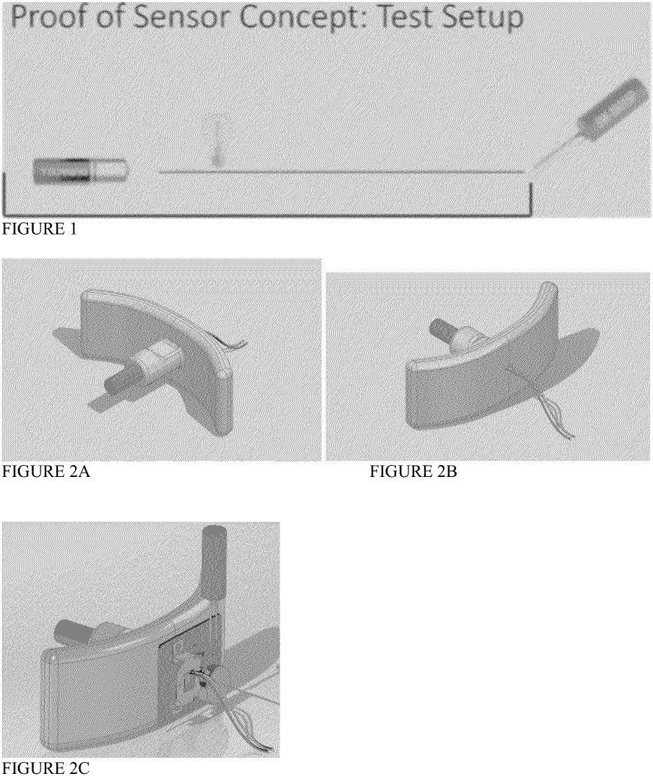

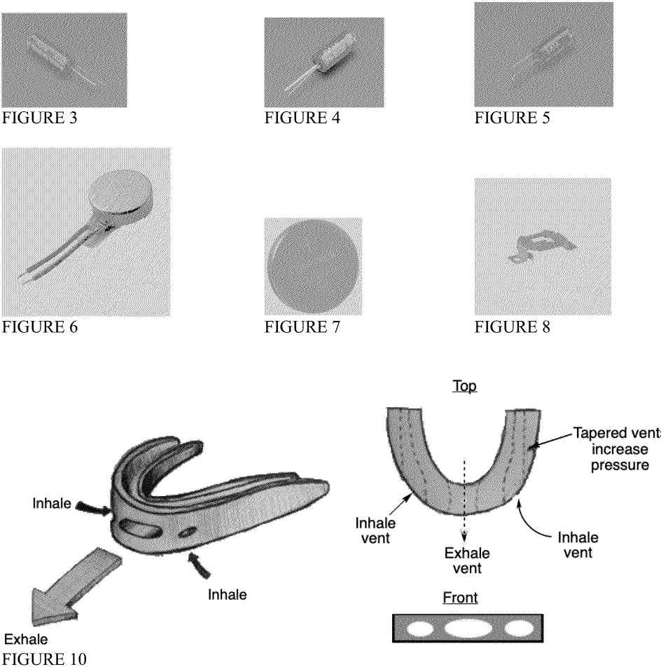

In one embodiment, the device uses an electric sensor to achieve the detection. In a preferred embodiment, an electronic vibration sensor is used. The vibration sensor works by sound waves vibrating an internal spring embedded within the sensor. When the spring vibrates, the switch is activated. The vibration causing switch is coupled with an electronic sensor. In an exemplary embodiment, the device comprises an electronic sensor connected with the vibration causing switch, then connected with an electronic power source (for example, a battery, FIG. 1). In a further exemplary embodiment, the present device is a device having a prototype shown as in FIGS. 2A-2C.

In an aspect, the vibration sensor can be a high sensitivity sensor, a medium sensitivity sensor, or a low sensitivity sensor. In a preferred aspect, the vibration sensor is a high sensitivity sensor. The high-sensitivity vibration sensor effectively detects various movements within the mouth, while effectively filtering out general head movements that may occur while sleeping. Filtering out generic head movements while sleeping prevents false triggering of the vibration motor.

In one embodiment, the device of the present invention comprises a Fast Vibration Sensor Switch (easy to trigger): These spring-vibration switches are high sensitivity non-directional vibration induced trigger switches. Inside is a very soft spring coiled around a long metal pin. When the switch is moved, the spring touches the center pole to make contact. When there is motion, the two pins will act like a closed switch. When everything is still, the switch is open. In an exemplary embodiment, the Fast Vibration Sensor Switch comprises the following technical details: Maximum Voltage: 12V; Contact Resistance: <10 ohm; Contact Time: 2 ms; Maximum Current: 20 mA; Insulation Resistance: >10 M ohm; Temperature Range: -40 to 80° C.; Operating lifespan: 100,000 cycles; Diameter: 5 mm/0.2", Height (w/ pins): 25 mm/1", Height (w/o pins): 15 mm/0.6", Weight: 0.3 g.

In another embodiment, the device of the present invention comprises a Medium Vibration Sensor Switch: These spring-vibration switches are medium sensitivity non-directional vibration induced trigger switches. Inside is a medium hardness spring coiled around a long metal pin. When the switch is moved, the spring touches the center pole to make contact. Accordingly, when there’s motion, the two pins will act like a closed switch. When everything is still, the switch is open. In an exemplary embodiment as shown in , the Medium Vibration Sensor Switch comprises the following technical details: Maximum Operating Temperature: 260° C. ± 10° C.; Contact Time: 2 -2.5 m; Diameter: 5 mm / 0.2"; Height (w/ pins): 23 mm / 0.9"; Height (w/o pins): 11 mm / 0.4"; Weight: 0.2 g.

In yet another embodiment, the device of the present invention comprises a Slow Vibration Sensor Switch (Hard to trigger): These spring-vibration switches are low sensitivity non-directional vibration induced trigger switches. Inside is a stiff spring coiled around a long metal pin. When the switch is moved, the spring touches the center pole to make contact. Accordingly, when there’s motion, the two pins will act like a closed switch. When everything is still, the switch is open. In an exemplary embodiment, the Slow Vibration Sensor Switch comprises the following technical details: Maximum Voltage: 12 V; Contact Resistance: <10 ohm; Contact Time: 2 ms; Maximum Current: 20 mA; Insulation Resistance: >10 M ohm; Temperature Range: -40 to 80° C.; Operating lifespan: 100,000 cycles; Diameter: 5 mm/0.2", Height (w/ pins): 21 mm/0.8"; Height (w/o pins): 11 mm/0.4", Weight: 0.30 g.

In more exemplary embodiments, the present device comprises the following components:
Digi-Key Part Number: 1670-1082-ND; Manufacturer: Jinlong Machinery & Electronics, Inc.; Manufacturer Product Number:C0827B005F; Supplier: Jinlong Machinery & Electronics, Inc.; Description: VIBRATION ERM MTR 12000 RPM 3V; Detailed Description- DC Motor Vibration, ERM 12000 RPM - - 3VDC.
Digi-Key Part Number: P031-ND; Manufacturer: Panasonic - BSG; Manufacturer Product Number:CR-1025BN; Supplier: Panasonic - BSG; Description: BATTERY LITHIUM 3V COIN 10MM; Detailed Description: Coin, 10.0 mm Lithium Manganese Dioxide 3 V Battery Non-Rechargeable (Primary).
Digi-Key Part Number: 36-3030-ND; Manufacturer: Keystone Electronics; Manufacturer Product Number: 3030; Supplier: Keystone Electronics; Description: BATTERY RETAINER COIN 10MM SMD; Detailed Description: Battery Retainer Coin, 10.0 mm 1 Cell SMD (SMT).

In another aspect, the device of the present invention is an oral device, with a probe positioned inside a subject’s mouth. In a further aspect, the device is a wearable device.

The present device can be used in detecting tongue or mouth movement associated with bruxism and/or sleep apnea comprising steps of 1) putting the device in to the mouth of a subject, and 2) collecting signals shown in the device.

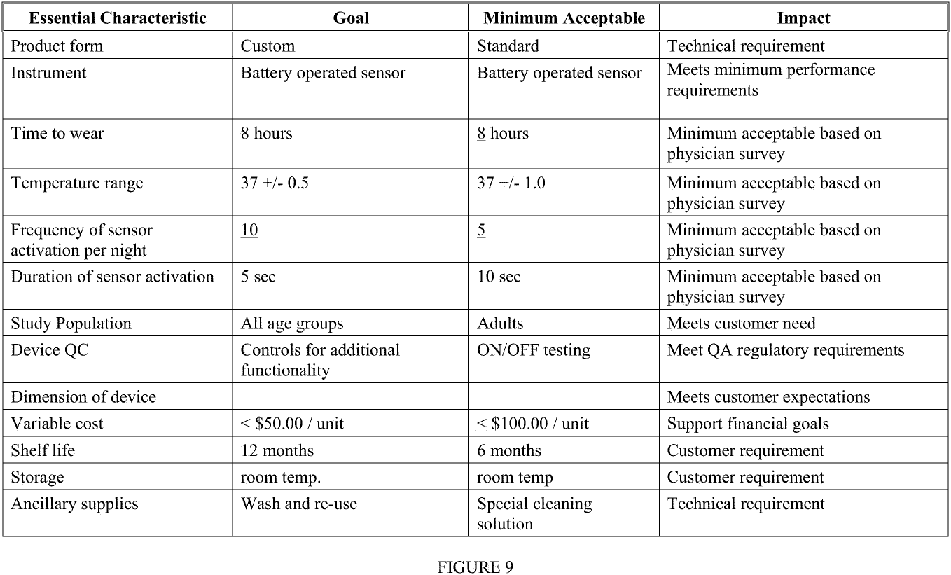

The present invention is also directed to a method of treating sleep apnea with the present device. The device is intended to detect the presence of tongue and other oral muscles and become activated to alert patients under certain conditions such as sleep apnea and/or tooth grinding during sleep. Performance Characteristics are summarized in FIG. 9.

The device of the present invention can be used not only to detect sleep apnea, but also treat sleep apnea. The present invention included a system for treating obstructive sleep apnea in a patient. The system comprises a sensing component and a stimulation component. In one embodiment, the sensor comprises one or more wireless sensors for collecting one or more vital signs of the patient and the muscle movement (such as tongue muscle movement) signals. The sensors for detection muscle tongue movement are the sensing devices of the present invention described above. The sensing component is preferably in wireless communication with a control system, and wherein the stimulation component comprises (i) an implantable body configured to deliver energy to one of a nerve or muscle, and (ii) a wearable appliance inductively coupled to the implanted body, the wearable portion configured to receive signals from the control system.

In an exemplary embodiment, the sensing component is placed to a patient inside of mouth. The sensor component detects tongue movements (e.g. moving forward in the mouth) of the patient during sleep. Next, the sensor component transmits a signal (either mechanical or electrical) to the stimulation component of the device, which is placed either inside or outside of the patient’s mouth. The stimulation component sends a stimulation signal to the tongue muscle which causes the patient’s tongue to move back in the mouth. In this way, the present system can both detect sleep apnea in a patient and send signals to the patient’s togue muscle to correct the tongue movement caused by sleep apnea. As a result, the sleep apnea symptoms can be alleviated. In a preferred embodiment, the disease can be cured.

The present invention is directed to a method of detecting or treating sleep apnea with the system disclosed herein.

### EXAMPLES

### Example 1

### Forefront Medical - Sleep Apnea Mouth Guard Sensor

### Part I: Introduction

Forefront Medical develops a wearable oral device that can detect the tongue movement associated with sleep apnea and bruxism via an electronic sensor or mechanical action. The following report discusses the proposed solution and the feasibility testing data of its ability to detectvarious mouth movements.

The first step was to explore the various options of sensors. The ideas were grouped into two categories: (a) mechanical based and (b) electrical based. The ideas generated in each category are explained in the following section.

### A. Mechanical Sensor

A brainstorming session was conducted to arrive at potential mechanical solutions where no electronics are used, and the entire system could fit inside the mouth. The ideas are explainedbelow.

### 1. Tongue Trap

This concept is based on the idea that there could be a specific geometry that would hold the tongue in a specific orientation such that it obstructs the tongue from sliding to the back of the throat. The specific design could take the form of a physical guard that sits at the back of the throat to keep the tongue in the front of the mouth. The benefit ofthis design is that it would be very cheap, fit entirely in the mouth, and could work with the current form factor. The drawbacks are that it would prove difficult to account for different tongue and mouth sizes and would most likely require custom fitting for each user. Furthermore, should the tongue slip past the guard, there would be no physical alarm to alert the user and has the potential of being uncomfortable with material beingplaced in the back of the throat close to the uvula. Ultimately, it was decided that the difficulties and drawbacks of the design are too significant to move further.

### 2. Flavored Guard

This idea is centered around the mouth guard being given a bad flavor so that when the tongue slides to the back of the throat, and contacts the guard, the user would be stirredawake due to the flavor of the mouth guard. The benefits of this design are in its cost and simplicity. Like the first design, designing a product that could account for various mouth and tongue sizes and the specific movements of the tongue, would be impossibly difficult to account for and was therefore ruled out. Furthermore, the risk of allergies is present with any flavors.

### 3. Continuous Positive Airway Pressure (CPAP)

CPAP functions by pushing about 10cmH2O air pressure through airways to keep them open. One can mechanically design tapered airways to increase air pressure upon inhale to mimic the pressure required to keep airways open. Airways integrated into moldable mouthguard and positioned to aim at back of throat. The “vents” could be one-way valves to make exhalation easier and focus the airstream upon inhalation. One can also add nose cover to ensure proper inhalation pressure. An exemplar sketch of CPAP design can be found in FIG. 10.

### B. Electrical Sensor

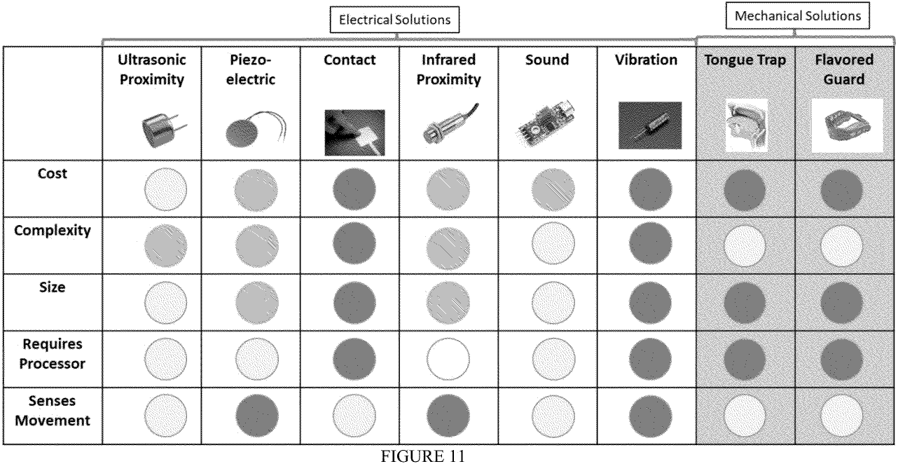

Various sensors were researched to determine which component would be the best for this application. See FIG. 11. Each sensor will be explained in the section below.

### 1. Ultrasonic Sensor

The ultrasonic sensor could work in this application by transmitting an ultrasonic soundwave toward the tongue and determine the distance of the tongue from the front of the mouth. The primary downside of this sensor is in its cost, size, and complexity of the circuit. The bulk of the components would have to live outside the mouth and would require signal processing to determine at what point the sensor needs to send a response signal to the vibrating motor. Considering this, the costs far outweigh the benefits.

### 2. Piezo-Electric Sensor

The piezo-electric sensor works by processing pressure applied to the sensor and sending a signal when the pressure is released. Its application within this product wouldbe to place it under the tongue so that when the tongue is at the front of the mouth applying downward pressure on the sensor. When the tongue slides to the back of the throat and the pressure is no longer present, the sensor would trigger a signal. While small and inexpensive, the sensor would require signal processing which drastically increases the size of the circuit board. Furthermore, a larger study would have to be conducted to determine the range of pressures applied by tongues. In the end, the complexity of determining that acceptable pressure range and the requirement of processing led to ruling this sensor out.

### 3. Contact Sensor

Like the piezo-electric sensor, the contact sensor works by simply detecting contact between a conductive material (in this case, the tongue) and the sensor itself. The cost,complexity, size, and signal processing are all ideal for this application. However, the main drawback is that the sensor does not detect overall movement, just movement off the sensor. The drawback here is that the saliva in the mouth, which is also conductive, could cause false positives in its detection.

### 4. Infrared Proximity Sensor

The infrared proximity sensor works by detecting the proximity of the tongue to the sensor itself. While the cost, size, and complexity are all favorable, the sensor does require processing which would increase the overall size of the product and require most of the components to be located outside of the mouth.

### 5. Sound Sensor

The idea behind the sound sensor is that it would be able to detect the sound caused by snoring associated with sleep apnea and the sound caused by teeth grinding. After research, it was discovered that not all sleep apnea causes snoring so the results could be unreliable. Furthermore, the cost, complexity, size, and processing of the sensor are all undesirable.

### 6. Vibration Sensor

The vibration sensor works by sound waves vibrating an internal spring embedded within the sensor. When the spring vibrates, the switch is activated. The vibration sensor ultimately proved to be the most attractive option as it provides the smallest package, lowest cost, requires no processing, and was able to sense the movement of the mouth.

Upon consideration of the mechanical sensors and electrical sensors, it was determined that a vibration causing switch coupled with an electronical sensor was the best option due to its small size, simple circuit design, and effectiveness at detecting the movements associated with bruxism and sleep apnea. The design would combine a vibration sensor switch with vibration sensing, providing a compact, 2-in-1 solution. Because the vibration sensor switch is available in three sensitivity levels: high,medium, and low. All three sensitivities were tested to establish which sensitivity would best detect the desired mouth movements.

### Part II: Purpose

The purpose of this experiment is to determine the viability of the vibration sensor for its application in sensing mouth movements and which sensitivity of the sensor will best detectcritical mouth movements. A summary of the mouth movements considered for this test can be found in Table 1.

Table 1

The critical mouth movements considered for testing
Mouth Movement
Light tongue flicking
Hard tongue flicking
Nodding head quickly
Shaking head quickly
Grinding teeth
Tongue sliding to back of throat
Swallowing
Nodding head slowly
Shaking head slowly

### Test Setup

This test uses:
5V Power Supply
Vibration sensor (Adafruit Product ID: 1766)
100 Ohm Resistor
Red LED
Bread board

FIG. 1 shows the electrical circuit for the test. There are 3 steps in testing: (1) Wrap vibration sensor wire leads in tape to protect against electrical shorting; (2) Place vibration sensor in mouth like a straw (floating in open air within the mouth; and (3) Perform each physical action and record if the LED turns on or not.

### Rationale for Pass-Fail Criteria

If the LED turns on via the vibration sensor being triggered, then it is conclusive that the physical motion has triggered the switch. The movements that should not trigger the switch are swallowing, nodding head slowly, and shaking head slowly as these are representative of normal sleep movements that should not be detected (Table 2).

Table 2

Pass/Fail criteria for vibration sensor testing

| Item Number | Requirement | Pass/Fail Criteria |
|---|---|---|
| 1 | Light tongue flicking | LED Turns on = Pass |
| 2 | Hard tongue flicking | LED Turns on = Pass |
| 3 | Nodding head quickly | LED Turns on = Pass |
| 4 | Shaking head quickly | LED Turns on = Pass |
| 5 | Grinding teeth | LED Turns on = Pass |
| 6 | Tongue sliding to back ofthroat | LED Turns on = Pass |
| 7 | Swallowing | LED Stays off = Pass |
| 8 | Nodding head slowly | LED Stays off = Pass |
| 9 | Shaking head slowly | LED Stays off = Pass |

### Results

As shown below in Tables 3-5, the highest sensitivity vibration sensor outperformed the medium and low sensitivity sensors. The high-sensitivity vibration sensor effectively detected various movements within the mouth. The high-sensitivity sensor also effectively filtered out general head movements that may occur while sleeping. Filtering out generic head movements while sleeping prevents false triggering of the vibration motor. This saves battery life by running the motor less often and avoids waking the patient up unnecessarily.

Table 3

Results of the test procedure for Highest Sensitivity

| Item Number | Requirement | Pass/Fail | LED Condition |
|---|---|---|---|
| 1 | Light tongue flicking | Pass | On |
| 2 | Hard tongue flicking | Pass | On |
| 3 | Nodding head quickly | Pass | On |
| 4 | Shaking head quickly | Pass | On |
| 5 | Grinding teeth | Pass | On |
| 6 | Tongue sliding to backof throat | Pass | On |
| 7 | Swallowing | Pass | Off |
| 8 | Nodding head slowly | Pass | Off |
| 9 | Shaking head slowly | Pass | Off |

Table 4

Results of the test procedure for Medium Sensitivity

| Item Number | Requirement | Pass/Fail | LED Condition |
|---|---|---|---|
| 1 | Light tongue flicking | Fail | Off |
| 2 | Hard tongue flicking | Pass | On |
| 3 | Nodding head quickly | Fail | Off |
| 4 | Shaking head quickly | Fail | Off |
| 5 | Grinding teeth | Fail | Off |
| 6 | Tongue sliding to backof throat | Fail | Off |
| 7 | Swallowing | Pass | Off |
| 8 | Nodding head slowly | Pass | Off |
| 9 | Shaking head slowly | Pass | Off |

Table 5

Results of the test procedure for Low Sensitivity

| Item Number | Requirement | Pass/Fail | LED Condition |
|---|---|---|---|
| 1 | Light tongue flicking | Fail | Off |
| 2 | Hard tongue flicking | Fail | Off |
| 3 | Nodding head quickly | Fail | Off |
| 4 | Shaking head quickly | Fail | Off |
| 5 | Grinding teeth | Fail | Off |
| 6 | Tongue sliding to backof throat | Fail | Off |
| 7 | Swallowing | Pass | Off |
| 8 | Nodding head slowly | Pass | Off |
| 9 | Shaking head slowly | Pass | Off |

### Analysis

Based on the results shown in Table 3-5, the high sensitivity vibration sensor is a great candidate for detecting movements within the mouth while filtering out generic head movementsduring sleep. It is unknown whether this solution will work on all patients, as everyone has different sleep patterns and movements and may have different reactions during an episode of sleep apnea. It is recommended that a functional prototype be built to test on patients and gather more data.

It is also recommended that the PCB primarily exist outside of the mouth as there are significantsafety risks associated with placing the battery inside the mouth. While there are options to explore for having the battery located in the mouth (such as titanium shielding), most of the options will drastically increase the cost and size of the device. As such, a custom mouthpiece be developed to appropriately reduce safety risks, determine the proper PCB layout, and design a housing that will optimize performance and safety to user. A prototype design of Sleep Apnea Mouth Guard Sensor device is shown in FIG. 2.

### Conclusion

The study results provide insight into the vibration sensor being an excellent candidate for detecting the specific mouth movements associated with sleep apnea and bruxism. The vibration sensor effectively filters out normal movement such as slowly shaking or nodding the head. Meanwhile, the vibration sensor can effectively detect teeth grinding and tongue flicking movements. When intentionally sliding the tongue to the back of the throat, the sensor was unintentionally flicked by the tongue, which lit up the LED. The movement of the tongue to the back of the throat can vary from person to person, and therefore may or may not be detected.

The vibration sensor switch has many desirable qualities for this application:
Low cost
Ease of electronic implementation
2-in-1 vibration sensor + switch that significantly simplifies electronic design
Ability to sense various movements within the mouth, while filtering out common headmovements
No need for electronic signal processing to function properly.

1. A sensor device for detecting movement of mouth or tongue in a subject associated with bruxism and/or sleep apnea, said device comprising:
a sensing component adapted to collecting one or more vital signs of the subject and the muscle movement signals; and
a stimulation component configured to filtering mouth or tongue movement not associated with bruxism and/or sleep apnea and deliver stimulating energy to a nerve or muscle in the subject’s mouth.

2. The device of claim 1, wherein said sensing component is an electronic sensor.

3. The device of claim 2, wherein said electronic sensor is a vibration sensor.

4. The device of claim 3, wherein said vibration sensor is coupled with a vibration causing switch that is connected with a power source.

5. A method of detecting tongue or mouth movement associated with bruxism and/or sleep apnea in a subject, said method comprising:
placing a device of claim 1 into the mouth of the subject;
collecting signals using the device; and
determining tongue or mouth movement associated with bruxism and/or sleep apnea in said subject using said collected signal.

6. A system for treating obstructive sleep apnea in a patient in need thereof, the system comprising a sensing component and a stimulation component, wherein the sensing component comprises one or more wireless sensors for collecting a vital sign and/or muscle movement signal of the patient, the sensing component being in wireless communication with a control system, and wherein the stimulation component comprises (i) an implantable body configured to deliver stimulating energy to a nerve, muscle, or combination thereof, and (ii) a wearable appliance inductively coupled to the implanted body, the wearable portion configured to receive signals from the control system.

7. The system of claim 6, where the vital sign is selected from the group consisting of blood oxygen, respiration rate, heart rate, and a combination thereof, wherein the muscle movement includes tongue muscle movement.

8. The system of claim 6, wherein the wearable portion is a dental appliance comprising a rechargeable battery, a pulse Hebert, and a means for inductively delivering electronic Ferry to the implantable body.

9. The system of claim 6, wherein the means of inductively delivering energy is a transmitter coil.

10. The system of claim 9, wherein the implantable body comprises a receiver coil for receiving energy from the wearable portion.

11. The system of claim 10, wherein the implantable body is configured to deliver stimulating energy to a hypoglossal nerve.

12. The system of claim 6, wherein the wearable portion is a dermal device for positioning on the patient’s skin, and wherein the dermal device comprises a means for inductively delivering energy to the implantable body.

13. The system of claim 12, wherein the implantable body is configured to deliver stimulating energy to a geniohyoid muscle.

14. The system of claim 13, wherein the implantable body comprises a means for wirelessly receiving energy from the dermal device, and wherein the implantable device further comprises an insulating disc.

15. The system of claim 14, wherein the control system having a first member of a pair of inductive power transfer coils, a wearable apparatus having a second member of a pair of inductive power transfer could, a rechargeable battery, and a pulse generator, wherein the wearable apparatus is configured to wirelessly deliver energy to the implantable body upon receipt of a signal inductive of a sleep apnea event; and wherein the implantable body is configured to transfer the energy received from the wearable apparatus to a hypoglossal nerve or a geniohyoid muscle positioned in proximity.

16. The system of claim 6, wherein the wearable appliance is a dental appliance adapted for placement over the patient’s lower teeth.

17. The system of claim 16, wherein the dental appliance is bite splint or retainer.

18. The system of claim 16, wherein the wearable apparatus further comprises means for receiving control signals from a control system communicatively coupled thereto.

19. The system of claim 6, where’s the control system comprises a processor, a memory, and a wireless communications module, the control system configured to (i) receive signals from one or more wireless sensors, (ii) process the signals to determine if a sleep apnea event has occurred or will occur, and (iii) send control signals to the wearable apparatus.

20. The system of claim 19, wherein the control system is embedded within the dental appliance.
